(19) United States
(12) Patent Application Publication
(10) Pub. No.: US 2003/0028093 A1
(43) Pub. Date: Feb. 6, 2003

(54) Exponential decay modeling in spectroscopy

(76) Inventors: Yong Ke, Belmont, MA (US); Perry Renshaw, Arlington, MA (US); Steven Lowen, Burlington, MA (US)

Correspondence Address: CHARLES H. SANDERS, Fish & Richardson P.C., 225 Franklin Street, Boston, MA, 02110-2804, US

(21) Appl. No.: 10/126,303

(22) Filed: Apr. 19, 2002

Related U.S. Application Data
(60) Provisional application No. 60/285,449, filed on Apr. 20, 2001.

(51) Int. Cl. A61B 5/05

(52) U.S. Cl. 600/410

(57) ABSTRACT

The invention permits measurement of changes in chemical concentrations where the chemicals have the same resonance peak in magnetic resonance spectroscopy. Using this approach, MRS techniques with higher resolution can be used, even though they fail to resolve chemicals of interest into different peaks. The invention can be applied to diagnose diseases, e.g., metabolic disorders, and to measure time-dependent chemical response to sensory or pharmaceutical stimuli.

### CROSS-REFERENCE TO RELATED APPLICATIONS

This application claims priority to U.S. Provisional Patent Application Serial No. 60/285,449, filed on Apr. 20, 2001, which is incorporated by reference herein in its entirety.

### STATEMENT AS TO FEDERALLY SPONSORED RESEARCH

This invention was made with Government support under National Institute on Drug Abuse grant DA09448 and National Institute of Mental Health grant MH57520. As a result, the Government has certain rights in the invention.

### TECHNICAL FIELD

This invention relates to magnetic resonance spectroscopy (MRS).

### BACKGROUND

MRS uses the fact that atomic nuclei spin to collect spectroscopic data. If the number of protons in a nucleus is even, their spins will cancel; however, if there is an odd number, there will be a net spin that can be used to conduct MR spectroscopy, see, e.g., U.S. Pat. No. 5,397,987, which is incorporated by reference herein. When a sample is placed in a main magnetic field, its nuclei align in the direction of the field (i.e., along the “magnetization axis”); the orientation of the nuclei can be represented by a magnetization vector, see, e.g., Horowitz, MRI Physics for Radiologists: A Visual Approach, 1995, which is incorporated by reference herein. These spinning nuclei can precess in a conical manner around the magnetization axis, generally out-of-phase with respect to each other. To induce in-phase spinning at the resonance frequency of particular nuclei, e.g., hydrogen or phosphorus nuclei, a radio frequency (RF) excitation pulse is broadcast at that resonance frequency. This RF pulse also causes the nuclei to rotate with respect to the magnetization vector created by the main magnetic field, see, e.g., Horowitz, MRI Physics for Radiologists: A Visual Approach, 1995, incorporated by reference herein. After the RF excitation pulse, RF free induction decay (FID) signals are collected, and used to generate NMR spectra.

An one-dimensional (1-D) NMR spectrum resolves resonance peaks of the chemicals in the test sample being evaluated along a chemical shift axis. A given chemical in the test sample has one or more spectroscopic components at one or more chemical shifts, and the concentration of this chemical in the test sample is represented by the intensity of these components. If two or more chemicals have spectroscopic components at the same chemical shift, then the intensity of the resonance peak at this point along the chemical shift axis reflects the aggregate contribution from each of these chemicals. If a given peak in the MR spectrum represents the spectroscopic component from only a single chemical, a change in the concentration of that chemical can be measured by calculating a change in the intensity at that resonance peak. If a given peak represents spectroscopic components from more than one chemical, measuring the change in intensity of that peak measures the aggregate change in concentration of all of the chemicals that contribute to that peak.

There are two major forms of relaxation, or decay, in MRS, see, e.g., E. Fukushima and S. Roeder, Experimental Pulse NMR—A Nuts and Bolts Approach, 1981, incorporated herein by reference. One form of relaxation is “de-phasing” of the nuclear spins over time after the RF pulse. This phenomenon is known as “spin-spin relaxation,” and the time constant for this spin-spin relaxation is labeled T₂. The relaxation phenomenon is exponential and is generally described by e^{-t}^{ₑ}^{/T}^{₂} where tₑ is the time after the RF excitation pulse. The other form of relaxation is the realignment of the magnetization vectors along the main magnetic field. This is known as “spin-lattice relaxation,” and the time constant for this spin-lattice relaxation is labeled T₁. This relaxation phenomenon is also exponential.

### SUMMARY

In one aspect of the invention, a test sample comprising two different chemicals is subjected to a magnetic field and an RF excitation pulse. In data acquired from excited nuclei, the two different chemicals have spectroscopic components at substantially the same chemical shift. The data are processed using information about a magnetic resonance relaxation property of each of the two chemicals to obtain concentration information about each of the two chemicals.

Advantages of this aspect of the invention include the following. The invention uses the fact that different chemicals can have different relaxation times to distinguish between or among chemicals that would otherwise be indistinguishable because they overlie one another at the same resonance peak at a common chemical shift. Because of the different relaxation times, each chemical's respective contribution to the intensity of the resonance peak at any echo time is reduced by a different amount, and this difference affords a means of determining, e.g., changes in the concentrations of the chemicals. The technique may be used for diagnostic purposes, e.g., to measure differences in chemical concentrations between an individual in a healthy state and the same, or a different, individual in a diseased state. In addition, the method may be used to evaluate the efficacy of a therapeutic technique by assessing changes in chemical concentrations over the course of treatment.

Embodiments of this aspect of the invention include one or more of the following features. The test sample can comprise more than two chemicals, each exhibiting substantially the same chemical shift. The relaxation property used to calculate the concentration information can be the spin-spin relaxation property, or the spin-lattice relaxation property. The concentration information can be calculated by using reference information about the concentration of each of the chemicals (e.g., information about the concentration of the two chemicals in the test sample at a different time, or in a different test sample), for instance to determine changes in concentration relative to the reference. The test sample can be brain tissue, and one of the chemicals of interest can be the phosphorylated form of another of the chemicals, e.g., phosphocreatine and creatine, and the method can be used to diagnose a metabolic disorder (e.g., phosphocreatine and creatine are both implicated in metabolic processes). The time constant for T₂ relaxation for phosphocreatine is between about 115 and 170 milliseconds, and the time constant for T₂ relaxation of creatine is between about 255 and 335 milliseconds. The chemical shift can be at about 3.08 ppm. The RF excitation pulse can be selected to excite ₁H nuclei in the test sample. This is useful because ¹H MRS generally provides a high level of sensitivity and accuracy. Thus, rather than use a less-sensitive or less-accurate MRS sequence to examine the chemicals independently (e.g., using ³¹P MRS, to obtain MR data for the phosphorylated chemical but not the non-phosphorylated form), with this aspect of the invention ¹H MRS can be used to examine both chemicals.

Another aspect of the invention includes a processor having an input for magnetic resonance spectroscopy data and a calculator for obtaining concentration information about each of two different chemicals that have spectroscopic components at substantially the same chemical shift in the magnetic resonance spectroscopy data, wherein the calculator employs information about a magnetic resonance relaxation property of each of the two chemicals. Yet another aspect includes a computer-readable storage medium comprising a program that is used by a processor to receive magnetic resonance spectroscopy data and obtain concentration information about each of two different chemicals that have spectroscopic components at substantially the same chemical shift in the magnetic resonance spectroscopy data by employing information about a magnetic resonance relaxation property of each of the two chemicals.

In another aspect of the invention, a test sample having at least two chemicals that have spectroscopic components at substantially the same chemical shift is subjected to a MRS sequence. FID signals are acquired after an echo time, tₑ, and the intensity of the resonance peak at the chemical shift, S(tₑ), is measured using these FID signals. A reference intensity S_{ref}(tₑ) is also determined, as are relationship(s) between or among the chemicals and a relaxation time constant Tᵢ for each of the chemicals. Changes in the concentrations of each of the chemicals relative to the reference intensity S_{ref}(tₑ) are calculated using the reference intensity S_{ref}(tₑ), the intensity S(tₑ), the relationships between the chemicals, and the relaxation time constants Tᵢ.

Embodiments of this aspect of the invention include one or more of the following features. Changes in the concentrations of each of the at least two chemicals can be determined from the relationship

$$Δ S ( {t}_{e} ) = {\sum }_{i = 1}^{n} Δ {S}_{i} {e}^{- {t}_{e} / {T}_{i}}$$

wherein ΔS(tₑ)=S(tₑ)−S_{ref}(tₑ); n is the number of the at least two chemicals, ΔSᵢ is a change in a contribution of each chemical to the intensity of the resonance peak at the chemical shift from a known reference value, Sᵢ^{ref}, to a value, Sᵢ, such that ΔSᵢ=Sᵢ−Sᵢ^{ref}, Sᵢ₊₁ is a function of Sᵢ for i=1 to n−1, and Sᵢ is a function f of Cᵢ, wherein Cᵢ is the concentration of each chemical, such that ΔCᵢ, the change in concentration, is given by the inverse off according to the equation ΔCᵢ=f⁻¹ (ΔSᵢ). Using this multi-exponential decay model, this method provides measurement of the changes in the concentrations of chemicals contributing to the same resonance peak.

In another aspect of the invention, a test sample having at least two chemicals that have spectroscopic components at substantially the same chemical shift is subjected to two MRS sequences, one at t₁ and the other at t₂. FID signals are acquired after an echo time, tₑ, for each sequence, and an intensity of the resonance peak at the chemical shift is determined at t₁, S(t₁,tₑ), and at t₂, S(t₂,tₑ), using the respective sets of FID signals. Relationship(s) between or among the chemicals are determined, as well as a relaxation time constant Tᵢ for each chemical. Changes in the concentrations of each of the chemicals between t₁ and t₂ are calculated using S(t₁,tₑ), S(t₂,tₑ), the relationships between the chemicals, and the relaxation time constants.

Advantages of this aspect of the invention include the following. This time-dependent technique is useful for determining changes in chemical concentration in response to stimuli, which can be indicative of metabolic response. The stimuli can be visual, auditory, or pharmaceutical.

Embodiments of this aspect of the invention include one or more of the following features. Changes in the concentrations of each of the at least two chemicals can be determined from the relationship

$$Δ S ( {t}_{e} ) = {\sum }_{i = 1}^{n} Δ {S}_{i} {e}^{- {t}_{e} / {T}_{i}} ,$$

wherein

ΔS(tₑ)=S(t₂,tₑ)−S(t₁,tₑ), n is the number of chemicals, ΔSᵢ is a change in a contribution of each chemical to the intensity of the resonance peak, Sᵢ(t), from a first value at t₁ to a second value at t₂ such that ΔSᵢ=Sᵢ(t₂)−Sᵢ(t₁), Sᵢ₊₁ is a function of Sᵢ for i=1 to n−1, and Sᵢ is a function f of Cᵢ, wherein Cᵢ is the concentration of each chemical, such that ΔCᵢ, the change in concentration, is given by the inverse of f according to the equation:

ΔCᵢ=f⁻¹(ΔSᵢ)

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a diagram of a apparatus for computing the changes in concentrations of chemicals that contribute to the intensity of the same resonance peak.

FIG. 2 is a diagram of a type of point-resolved echo spectroscopy sequence.

FIG. 3 is a graph of peak amplitude for the PCr/Cr peak fitted for mono-exponential and bi-exponential decay.

FIG. 4 is a graph of peak amplitude for the PCr/Cr peak, normalized to the peak for N-acetyl aspartate, for a period of photic stimulation.

FIG. 5 is a brain image showing the location of a voxel for analysis.

FIG. 6 is a graph of Cre (PCr and Cr) amplitude versus TE.

FIG. 7 is a stack plot of Cre amplitude versus chemical shift.

FIG. 8 is a graph of Cre amplitude fitted for mono-exponential and bi-exponential decay.

FIG. 9 is a table with the results of an experiment on cocaine-dependent subjects (Patient) and a healthy comparison group (Comparison).

FIG. 10 is a graph of the changes in PCr/tCr from baseline to final in cocaine-dependent subjects for both responder and non-responder groups.

### DETAILED DESCRIPTION

The Test and Data Processing Apparatuses

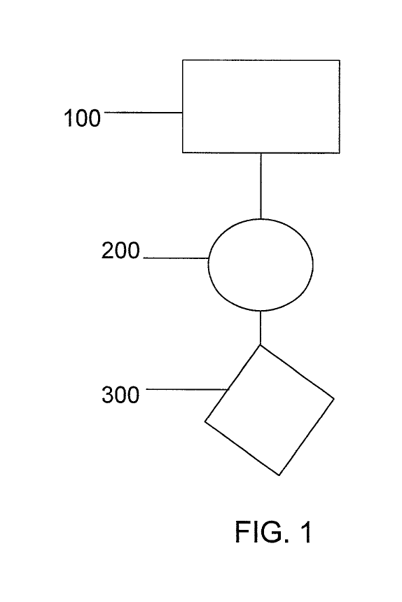

An apparatus for computing the changes in concentrations of each of a number of different chemicals that have the same chemical shift, and thus all contribute to the same resonance peak in a MR spectrum, is shown in FIG. 1. This apparatus contains a magnetic resonance scanner 100, a receiver 200, and a processor 300. The magnetic resonance scanner 100 subjects a test sample (the test sample can be an ex vivo sample, e.g., of dead tissue, or an in vivo sample, e.g., of living tissue such as brain tissue) with chemicals of interest to a MRS sequence, and the receiver 200 collects FID signals after certain echo times, tₑ, and converts them into electric signals. To calculate changes in the concentrations of the chemicals, the processor 300 computes intensities of the resonance peak for the particular echo times from the FID signals it receives as input from the receiver 200. The processor 300 runs a software routine (a program contained on a computer-readable storage medium that is loaded into RAM and executed by the processor) containing either a static or a time-dependent equation that uses these intensities to determine a change in the peak intensity and, using this change in peak intensity, to determine the changes in chemical concentrations.

MRS Sequence

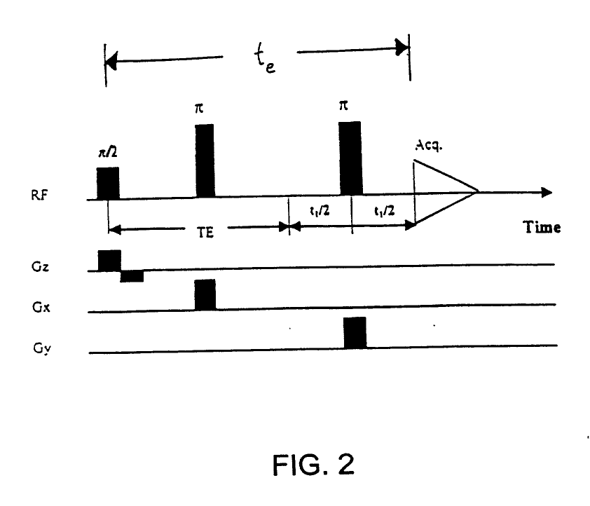

The magnetic resonance scanner 100 subjects the test sample to a point-resolved echo spectroscopy (PRESS) sequence. This sequence is shown in FIG. 2. In the figure, the RF line shows the RF pulses used in the sequence, while the Gz, Gx, and Gy lines respectively diagram the gradient pulses in the z, x, and y directions. The π and π/2 bars on the RF line denote 180° and 90° pulses, respectively. TE is the time in each sequence before t₁, and a data acquisition, Acq., occurs after each t₁. The total echo time before data acquisition is tₑ, which is equal to the sum of TE and t₁. In this sequence, there are two waiting periods, t_{1/2}, one before and one after the second π pulse. These sequences are repeated with a chosen repetition time (TR).

Data Acquisition and Processing

To analyze the chemical concentrations of a test sample, the test sample is subjected to the MRS sequence shown in FIG. 2 to acquire FID signals at one or more echo times tₑ. Intensities of the resonance peak at these echo times tₑ are measured, and S(tₑ) represents the intensity of the peak in the sample for a particular echo time. Since FID signals exponentially decay after the RF excitation pulse, the value of S(tₑ) is an exponential decay function of a maximum value S, where the amount of decay is determined by the echo time and the time constant for the decay, T; thus, S(tₑ)=Se^{-t}^{ₑ}^{/T}. Solving for the change in chemical concentration involves expressing the measured resonance peak intensities, S(tₑ), as a change from a known value, S_{ref}(tₑ) to provide a change in peak intensity ΔS(tₑ): ΔS(tₑ)=S(tₑ)−S_{ref}(tₑ). This method can be used to calculate changes in chemical concentrations relative to the reference value.

The reference value, S_{ref}(tₑ), can be obtained from any suitable source. For instance, the reference information can be data obtained from a prior spectroscopic analysis of the same test sample, and the present method would reveal changes in concentrations since the last test. As an illustration, if the “test sample” is a patient's brain tissue, the present method can be used to compare concentration levels of metabolites in the brain such as phosphocreatine and creatine at successive points in time, such as to evaluate the efficacy of treatment for metabolic disorders.

Alternatively, the reference information can be data obtained from a similar class of test samples. As an illustration, if the test sample were again brain tissue, the reference information can be obtained from a set of tests run on a patient known to be healthy. The present method can be used to compare the concentration levels of metabolites in the brain such as phosphocreatine and creatine between the “healthy patient” and the one under examination, such as to facilitate or otherwise aid in the diagnosis of metabolic disorders.

The reference information can alternatively be obtained from publications, such as data reporting “normal” concentration levels, in order to evaluate whether and the extent to which a patient deviates from the norm.

The contribution of each chemical to S, the peak intensity, is the sum of the individual contribution from each of the chemicals to that peak:

$$S = {\sum }_{i = 1}^{n} {S}_{i} ,$$

, where Sᵢ is a “contribution factor” for each chemical, and where i runs from one to the number of chemicals contributing to the same peak, n. For each chemical, there is a change in the contributor factor, ΔSᵢ, from some reference value, Sᵢ^{ref}, such that ΔSᵢ=Sᵢ−Sᵢ^{ref}, where

$${S}_{ref} = {\sum }_{i = 1}^{n} {S}_{i}^{ref} .$$

Thus, the change in maximum peak intensity, ΔS, is a sum over the changes in the contribution factors of all the chemicals:

$$Δ S = {\sum }_{i = 1}^{n} Δ {S}_{i} .$$

The contribution of each chemical to the peak intensity at an echo time, Sᵢ(tₑ), is the contribution factor, Sᵢ, multiplied by a factor to account for the exponential decay over the echo time tₑ: Sᵢ(tₑ)=Sᵢe^{-t}^{ₑ}^{/T}^{₁}, where Tᵢ is the spin-spin relaxation time constant T₂ for each chemical. (The spin-lattice relaxation time T₁ can alternatively be used.) The change in the peak intensity measured at an echo time, ΔS(tₑ), is the sum of the changes in the contribution factors of all the chemicals at that echo time, ΔSᵢ(tₑ), such that:

$$Δ S ( {t}_{e} ) = {\sum }_{i = 1}^{n} Δ {S}_{i} ( {t}_{e} ) .$$

. Since Sᵢ(tₑ)=Sᵢe^{-t}^{ₑ}^{/T}^{₁}, this relationship can be expressed as:

$$Δ S ( {t}_{e} ) = {\sum }_{i = 1}^{n} Δ {S}_{i} {e}^{- {t}_{e} / {T}_{i}} .$$

.

The method also involves determining a relationship between the contribution factor of each chemical, Sᵢ, and its concentration Cᵢ. A function f describes the relationship between the change in each contribution factor, ΔSᵢ, and the change in the chemical concentration, ΔCᵢ:ΔSᵢ=f(ΔCᵢ). Therefore, the change in chemical concentration, ΔCᵢ, is the inverse of this function, which is expressed as ΔCᵢ=f⁻¹(ΔSᵢ). Usually, f is a linear function.

The method also involves determining a relationship between or among the contribution factors, Sᵢ. Specifically, Sᵢ₊₁, is determined as a function of Sᵢ for i=1 to n−1. In some instances, the equation ΔSᵢ=f (ΔCᵢ) may be used to express the relationship between Sᵢ₊₁, and Sᵢ, such as if the relationship(s) between or among the chemicals can be expressed as chemical equations, such that Cᵢ₊₁ is a function g of Cᵢ for i=1 to n−1. An example of function g is ΔCᵢ₊₁=−ΔCᵢ, which reflects the underlying chemical phenomenon where each unit of Cᵢ is converted into a unit Cᵢ₊₁. Thus, for a linear function f, Sᵢ₊₁=f(g(Cᵢ)). This equation means that, if the concentration of one chemical is known, along with the equations relating the chemicals and the relationship between spectral intensity and concentration, the peak intensity can be predicted because all spectral contributions can be calculated. Therefore, a diagnostic technique can involve assessing the concentration of a chemical by some testing means, e.g., a blood, urine, or MR test, that provides the concentration of this chemical, and the present method can be used to measure peak intensity for that chemical and the other contributors. This peak intensity would reveal a deficiency or excess in the other chemicals that contribute to the same resonance peak, and that data could be used for diagnostic purposes.

As previously described, the overall change in peak intensity is a sum of the changes in all the individual contributions to this change:

$$Δ S ( {t}_{e} ) = {\sum }_{i = 1}^{n} Δ {S}_{i} {e}^{- {t}_{e} / {T}_{i}} .$$

. If the change in chemical concentration cannot practically be expressed as a direct function of all other quantities in its generalized form, using the equation ΔCᵢ=f⁻¹(ΔSᵢ), the equation to be solved can be rewritten as:

$$Δ S ( {t}_{e} ) = {\sum }_{i = 1}^{n} f ( Δ {C}_{i} ) {e}^{- {t}_{e} / {T}_{i}} .$$

This equation can be solved for ΔCᵢ because all other quantities are known or measured.

Generally, the foregoing calculations are simplified if the same echo time is used for each MRS sequence. However, it is possible to use different echo times for different sequences. If this is done, intensity values taken from the different echo times can be “scaled” to one another by using an exponential factor. For instance, an intensity taken at a first echo time, tₑ¹, can be scaled to a value at a second echo time, tₑ², by using an exponential factor tₑ²−tₑ¹, S(tₑ²)=S(tₑ¹)e^{-(t}^{ₑ}^{-t}^{ₑ}^{¹}^{)/T}.

The foregoing method can be used to determine the change in chemical concentration for each of a number of different chemicals. Consequently, it can be used for diagnostic purposes, e.g., where the chemical levels in a patient are compared to known levels in another healthy individual or to the patient's own levels at a previous time, e.g., an earlier visit.

The method can also be used to evaluate more rapid time-dependent changes in chemical concentration. Analyzing time-dependent changes is useful for many purposes, including assessing metabolic response to a sensory or pharmaceutical stimulus.

This approach introduces a variable for time, t, measured from some arbitrary starting point, such as the start of the experiment. Since the time-dependence of intensity is used to calculate the time-dependent change in concentrations, the measured intensity is written as a function of t and tₑ, yielding S(t,tₑ). 66 S(tₑ) is a change is S(t,tₑ) from a first time, t₁, to a second time, t₂, such that ΔS(tₑ)=S(t₂,tₑ)−S(t₁,tₑ). Similarly, ΔSᵢ represents a change in the contribution of each chemical to the intensity of the resonance peak from a known value at a first time, t₁, to a value at second time, t₂, such that ΔSᵢ=Sᵢ(t₂)−Sᵢ(t₁). Thus, ΔCᵢ represents a change in concentration over time, since it is equal to f⁻¹(ΔSᵢ).

### EXAMPLE 1

A time-dependent evaluation was conducted of the change in the concentrations of two metabolites in the brain, Cr and PCr, due to photic stimulation. PCr is a molecule that the body uses for energy, and it is converted into Cr via the creatine kinase reaction: PCr+ADP+H⁺ [symbol] Cr+ATP, see, e.g., Lehninger and Albert, Principles of Biochemistry, 1982, which is incorporated herein by reference. Therefore, the ability to measure changes in the concentrations of PCre and Cre is useful, as their concentrations are indicative of the metabolic activity in the tissue sample.

The peak intensity for these chemicals was modeled using a bi-exponential equation involving a T₂ decay constant for each chemical. To determine the T₂ for PCre and Cre, the sequence shown in FIG. 2 was applied to 14 human volunteers using a 1.5T GE SIGNA scanner. In the scanning sequence, tₑ ranged from 48 milliseconds to 678 milliseconds, in increments of 10 milliseconds. The repetition time, TR, was 2.64 seconds. In vivo MR spectra were acquired from a voxel in the left frontal lobe of their brains having a volume of 18.75 cm³. Eight averages were recorded for each brain spectrum. A phased array receiver was used for data acquisition, improving receiver sensitivity by approximately four times compared to the standard GE quadrature head coil.

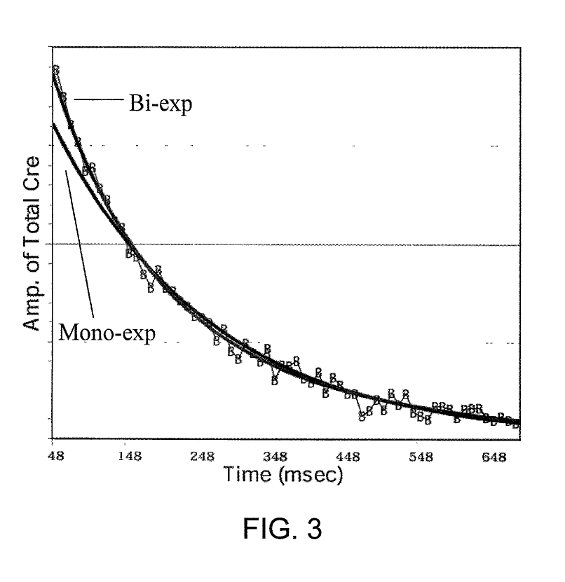

Using curve-fitting software, the tₑ dependence curves of the Cr resonance amplitudes from in vivo spectra were fit with models both of single exponential and bi-exponential decay as shown in FIG. 3, where Cre stands for the combined PCr and Cr contributions. FIG. 3 reveals that the bi-exponential model was a better fit, i.e., higher R². These studies revealed that PCr has a T₂ of 143.3+28.7 milliseconds and Cr has a T₂ of 297.1±40.7 milliseconds. This result indicated that the ¹H MRS signal of Cr and PCr is dependent on echo time, providing a means for resolving the changes in the concentrations of these chemicals.

A photic stimulation study was performed on eight volunteers. In this study, 150 water-suppressed spectra, plus four spectra without water suppression, were acquired using the sequence shown in FIG. 2 from a voxel of 5.12 cm³ located over the primary visual cortex in the right occipital lobe. Using a TR of 4 seconds and a tₑ of 272 milliseconds, 15 spectra were recorded every 2 minutes, with two averages in each spectrum. Total scan time was 20 minutes. Thus, the time parameter t ranged from 0 to 20 minutes, in increments of 2 minutes. Photic stimulation was performed using GRASS (Quincy, Mass.) goggles emitting 8 Hz flashing lights. The goggles were turned on for three separate two-minute periods beginning at the 6 minute, 12 minute and 18 minute marks during the scan.

Previous studies at 1.5 T and higher fields have shown that T₂ differences for the Cr resonance in gray and white matter are negligibly small, see, e.g., Frahm et al., Magnetic Resonance in Medicine, 38(4):551, 1997; Hetherington et al., Magnetic Resonance in Medicine, 32(5):565, 1994; Chen et al., Radiology, 173(2):521, 1989, which are all incorporated by reference herein. Therefore, the T₂ values obtained from the 18.75 cm³ in the left frontal lobe of the 14 subjects, 143.3±28.7 milliseconds for PCr and 297.1±40.7 milliseconds for Cr, were used as the relaxation time constant Tᵢ in this experiment.

It was assumed that PCr contributed 48% and Cr contributed 52% to the total Cr proton MRS signal at t=0 and tₑ=0, based on reports regarding their in vivo concentrations, see, e.g., Hoang et al., Neurology, 50(4):1033, 1998; Rothmnan et al., PNAS USA, 90(12):5662, 1993. Thus, S(t=0,tₑ=0) for PCr was 48% and S(t=0,tₑ=0) for Cr was 52%. Since f was merely a proportionality constant in this experiment, ΔSᵢ=kᵢΔCᵢ, where kᵢ is the proportionality constant, which was assumed to be 1 for both PCr and Cr; therefore, ΔSᵢ=ΔC. Thus, the initial concentration ratio of PCr to Cr was assumed to be 0.48 to 0.52.

Using this information, the change in the concentrations of PCr and Cr during the experiment, ΔC_{PCr} and ΔC_{cr}, respectively, were determined by solving the equation:

$$Δ S ( {t}_{e} ) = {\sum }_{i = 1}^{n} Δ {S}_{i} {e}^{- {t}_{e} / {T}_{2}^{i}} .$$

Superscripts, rather than subscripts are used to denote the T₂ values for each chemical to avoid confusion with the relaxation time constant for T₁ decay. Thus, T₂¹ is the T₂ value for chemical 1, and T₂² is the T₂ value for chemical 2. For two chemicals, this equation reduced to: ΔS(tₑ)=ΔS₁e^{-t}^{ₑ}^{/T}^{₂}^{¹}+ΔS₂e^{-t}^{ₑ}^{/T}^{₂}^{²}. Substituting the equation ΔSᵢ=ΔCᵢ, the solution equation was reduced to: ΔS(tₑ)=ΔC_{PCr}e^{-t}^{ₑ}^{/T}^{₁}+ΔC_{cr}e^{-t}^{ₑ}^{/T}^{₂}, where PCr is arbitrarily chosen to be chemical 1 and Cr is chemical 2.

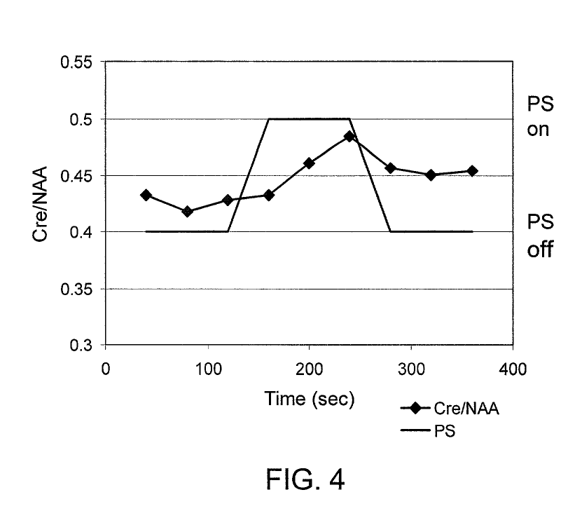

The creatine kinase reaction, PCr+ADP+H⁺ [symbol] Cr+ATP, provided the relationship between C₁ and C₂. As the concentration of either PCr or Cr increased, the concentration of the other experienced a corresponding decrease; therefore, ΔC₂=−ΔC₁ (and ΔS₂=−ΔS₁, since ΔSᵢ=ΔCᵢ). Using this relationship, the solution equation can be further reduced to: ΔS(tₑ)=ΔC_{PCr}(e^{-t}^{ₑ}^{/T}^{₂}^{¹}−e^{-t}^{ₑ}^{/T}^{₂}^{²}). Substituting the T₂ values obtained for PCr and Cr, the solution equation became: ΔS(tₑ)=ΔC_{PCr}(e^{-t}^{ₑ}^{/1143.3}−e^{-t}^{ₑ}^{/297.1}). Substituting for the tₑ value of 272 milliseconds, the equation was: ΔS(tₑ)=Δ_{PCr}(e^{-272/143.3}−e^{-272/297.1}). During periods of photic stimulation, the total peak intensity at the end of each two-minute stimulation period had a significant average increase of 11.1±3.5% over the signal intensity before stimulation. The increase during the first photic stimulation period is shown in FIG. 4, where PS stands for photic stimulation. In FIG. 4, the signal intensity for the PCr/Cr peak (together denoted as “Cre” in FIG. 4) is given relative to a reference peak for N-acetyl aspartate (NAA), since it is presumed to remain constant during the experiment. In addition, peak signal in all three stimulation periods demonstrate a linearly increasing pattern with an average slope of 3.6% per minute, as shown by the third to fifth data points in FIG. 4. This increase in the intensity of the resonance peak for PCr and Cr indicates conversion of PCr to Cr during photic stimulation, see, e.g., Murashita et al., Psychological Medicine, 30(1):107, 2000; Kato et al., Journal of the Neurological Sciences, 155(2):182, 1998; Chen et al., Magnetic Resonance in Medicine, 38(4):551, 1997, which are incorporated by reference herein.

Substituting for the 11.1% change in peak intensity, the solution equation becomes: 0.111Sₒ=ΔC_{PCr}(e^{-272/143.3}−e^{-272/297.1}),where Sₒ is the peak intensity before photic stimulation. Sₒ is expressed as the peak intensity based on the initial concentrations of PCr, C_{PCr}_{ₒ}, and Cr, C_{cr}_{ₒ}, as follows: Sₒ=C_{PCr}_{ₒ}e^{-t}^{ₑ}^{/T}^{₂}^{²}+ΔC_{Cr}_{ₒ}e^{-t}^{ₑ}^{/T}^{₂}^{²}. Substituting the appropriate values, Sₒ=0.48e^{-272/143.3}+0.52e^{-272/297.1}. Therefore, the solution equation becomes: 0.111(0.48e^{-272/143.3}+0.52e^{-272/297.1})=ΔC_{PCr}(e^{-272/143.3}−e^{-272/297.1}) Solving for ΔC_{PCr}, the equation reduces to: ΔC_{PCr}=−0.124.

Consequently, the 11.1±3.5% increase in total peak intensity over the entire photic stimulation period indicates a decrease of 25.9% in PCr concentration and an increase of 23.9% in Cr concentration. The 3.6% increase in signal intensity per minute corresponded to a 8.4% decrease in PCr per minute and a 7.8% increase in Cr per minute. This change is the result of higher metabolic activity in occipital cortex during photic stimulation. PCr was converted into Cr via the creatine kinase reaction, releasing energy. Since PCr has a shorter T₂ decay time than Cr, an increase in peak intensity indicates an increase in Cr concentration, i.e., faster metabolism, while a decrease in peak intensity indicates an increase in PCr concentration, i.e., a slowed metabolism.

### EXAMPLE 2

Nineteen cocaine-dependent (CD) subjects were assessed before and after eight weeks of treatment for cocaine dependence, and a comparison group of nine healthy volunteers was also studied on two occasions separated by 8 weeks. All CD subjects met the DSM-IV diagnosis of cocaine dependence and reported using cocaine on at least six occasions within the 28-day period prior to screening. Self-report of current cocaine use was substantiated with three urine specimens that were positive for the cocaine metabolite benzoylecsomine (BE) over a two-week period prior to entry into the study.

An Addiction Severity Index (ASI), see, e.g., McLellan et al., The Fifth Edition of the Addiction Severity Index, J. Substance Abuse Treatment, 9(3):119-213, 1992, which is incorporated by reference herein, was obtained prior to randomization and after eight weeks of treatment, and a quantitative urine toxicology screen for BE was obtained three times a week during screening and throughout the study. Response to treatment was assessed in two ways: by self-report (SR) and by urine toxicology report (UR). The response to treatment based on self report of cocaine use was determined by the number of days cocaine was used in the last thirty days prior to treatment, as reported on the ASI and compared to the ASI Follow-Up Questionnaire after eight weeks of treatment. A twenty-five percent drop in the number of days of reported cocaine use in a thirty-day period was considered to be SR positive. All quantitative urine BE levels obtained the week prior to initial treatment were averaged, as were all quantitative urine BE levels obtained after eight weeks of treatment. A twenty-five percent drop in mean urine BE levels from the first week to the last week was considered to be UR positive.

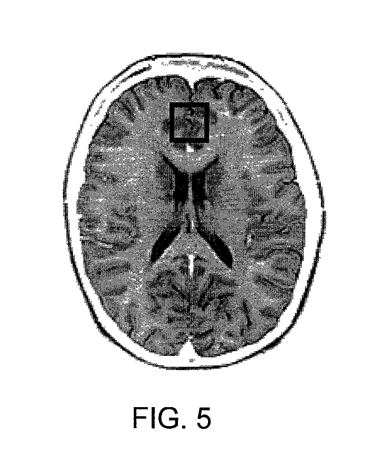

A modified PRESS MRS sequence as shown in FIG. 2 was implemented on a GE 1.5 Tesla SIGNA MR scanner. Localized spectra were acquired from the left anterior frontal lobe shown in FIG. 5 with a voxel size of 2.5×2.5×3 cm³. Other scan parameters included: TR=2.32 sec and 64 values of TE ranging from 48 msec to 678 msec. Each recorded FID had eight averages. Spectral bandwidth was 2000 Hz with 2048 data points.

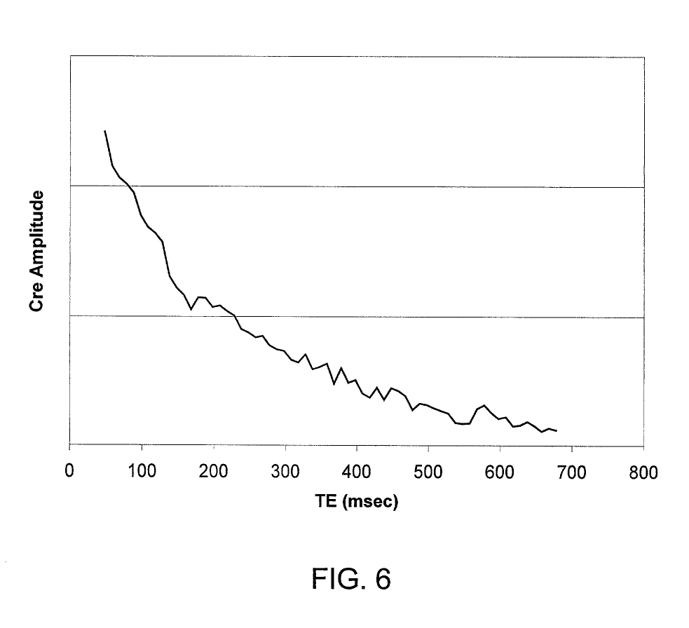

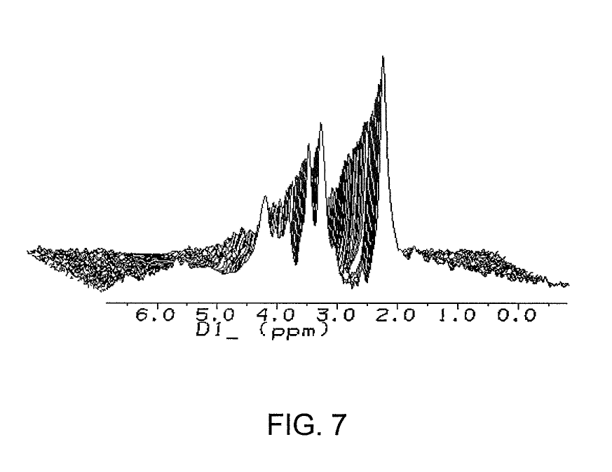

After fully automated spectral processing, signal amplitudes for the 3.0 ppm creatine resonance were extracted from each spectrum at each of the 64 TE values. Dependence of the Cre amplitude on TE is shown in FIG. 6, and a stack plot of the data versus chemical shift is shown in FIG. 7.

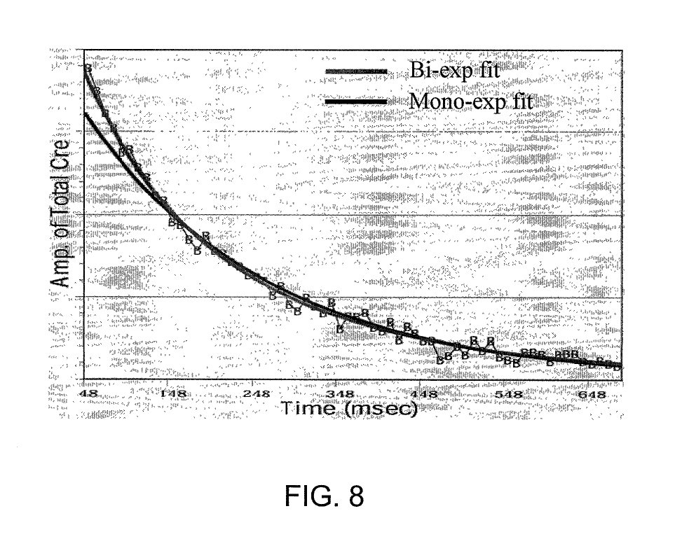

The Cre amplitude-TE curve obtained from each visit was fit to mono-exponential and bi-exponential models, with phosphocreatine to total creatine (PCr/tCr) initially set to 0.48. Random permutations of the residues from the mono-exponential model were also fit to the bi-exponential model, in order to obtain non-parametric significance values for the goodness of fit. Any results yielding significance values greater than 0.02 were eliminated, and only subjects with both visits meeting this significance criterion were included. A graph of the mono-exponential and bi-exponential fits to the Cre amplitude signal in vivo is shown in FIG. 8.

From the models, decay times were obtained for PCr from all the healthy comparison subjects, and the median was calculated; the same procedure was performed for Cr. These values were in turn used to estimate the relative concentrations of PCr and Cr for both groups. Overall, the PCr/tCr ratios obtained were similar to the 0.48 value employed in the initial bi-exponential fits, lending confidence to this starting value. Each Cre amplitude-TE curve was then fit for T₂ values with both mono-exponential and bi-exponential modes and statistically analyzed across groups.

T₂ relaxation decay curves for the Cre resonance at 3.0 ppm both for CD and comparison subjects clearly demonstrated a bi-exponential decay characteristic for most subjects (T₂ for PCr was 84 msec, T₂ for Cre was 287 msec). For both groups, only subjects with a significantly better fit (p<0.05) to a bi-exponential rather than a mono-exponential form were included. Estimated values for T₂ for PCr and T₂ for Cr were not significantly different between CD and comparison subjects.

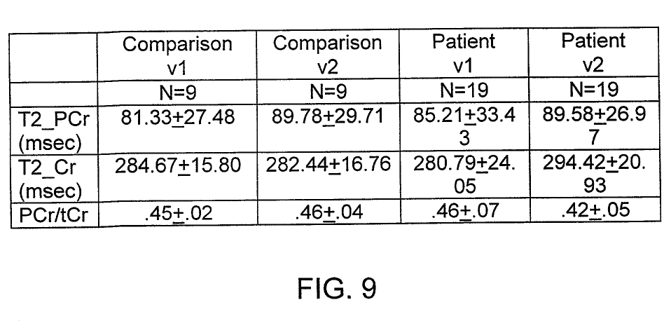

At baseline, mean PCr/tCr were 0.46 and 0.45 in the CD and comparison groups, respectively. After treatment, PCr/tCr ratios were 0.42 for CD subjects while the mean PCr/tCr ratio for the comparison group remained approximately the same, 0.46. These results are shown in the table in FIG. 9.

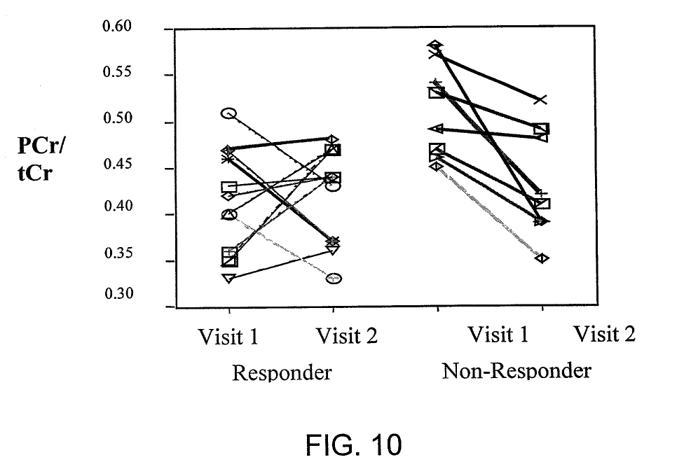

The mean difference in PCr/tCr from baseline to final was −0.03 in CD subjects and 0.01 in comparison subjects, although this result did not reach statistical significance. However, when CD subjects were divided into Responder/Non-Responder groups, there was a significant decrease in mean PCr/tCr with treatment in the Non-Responder group, while there was no significant change in mean PCr/tCr in the Responder group. FIG. 10 shows the changes in PCr/tCr from baseline to final in CD subjects for both responder and non-responder groups.

When CD subjects were divided by their drug treatment groups (venlafaxine, pramipexdle, and placebo), PCr/tCr values of all three groups decreased after treatment compared to their baseline values: −0.03 for venflaxine; −0.04 for pramipexdle; and −0.03 for placebo.

Analyses were done to see if there were significant differences between PCr/tCr in males and females at baseline and after treatment. At baseline, males (0.45±0.07) and females (0.50±0.08) did not differ in mean PCr/tCr. Additionally, the female group (−0.09±0.11) had a greater decrease in PCr/Cr over the course of treatment than the male group (−0.02±0.07).

A number of embodiments of the invention have been described. Nevertheless, it will be understood that various modifications may be made without departing from the spirit and scope of the invention. For example, the invention can be applied to assess the effects of drug treatment on metabolite concentrations in the brains of individuals undergoing pharmaceutical treatment for a brain disorder. Accordingly, other embodiments are within the scope of the following claims.

What is claimed is:

1. A magnetic resonance spectroscopy method comprising:
(a) subjecting a test sample comprising two different chemicals to a magnetic field;
(b) subjecting the test sample to an RF excitation pulse to excite nuclei in the test sample;
(c) acquiring data from excited nuclei in the test sample, wherein the two different chemicals in the test sample have spectroscopic components at substantially the same chemical shift; and
(d) processing the acquired data to obtain concentration information about each of the two chemicals, wherein the processing step employs information about a magnetic resonance relaxation property of each of the two chemicals.

2. The method of claim 1 wherein the test sample comprises at least three different chemicals.

3. The method of claim 2 wherein the at least three different chemicals in the test sample have spectroscopic components at substantially the same chemical shift.

4. The method of claim 3 wherein the processing step obtains concentration information about each of the at least three different chemicals in the test sample by employing information about a magnetic resonance relaxation property of each chemical.

5. The method of claim 1 wherein the magnetic resonance relaxation property is the spin-spin relaxation property.

6. The method of claim 1 wherein the magnetic resonance relaxation property is the spin-lattice relaxation property.

7. The method of claim 1 wherein the test sample comprises brain tissue.

8. The method of claim 1 wherein one of the chemicals in the test sample is phosphocreatine.

9. The method of claim 1 wherein one of the chemicals in the test sample is creatine.

10. The method of claim 1 wherein one of the two chemicals is a phosphorylated form of the other of the two chemicals.

11. The method of claim 1 wherein the RF excitation pulse excites ¹H nuclei in the test sample.

12. The method of claim 1 wherein the chemical shift is about 3.08 ppm.

13. The method of claim 1 further comprising the step of diagnosing a disorder by evaluating the concentration information.

14. The method of claim 1 further comprising the step of diagnosing a metabolic disorder by evaluating the concentration information.

15. The method of claim 1 wherein the processing step further employs reference information about the concentration of each of the two chemicals.

16. The method of claim 15 wherein the reference information comprises information about the concentration of the two chemicals in the test sample at a different time.

17. The method of claim 15 wherein the reference information comprises information about the concentration of the two chemicals in a different test sample.

18. The method of claim 15 wherein the concentration information about each of the two chemicals comprises information about a change in the concentration of each of the two chemicals relative to the reference information.

19. The method of claim 1 further comprising the step of diagnosing response of a cocaine-dependent subject to treatment by evaluating the concentration information.

20. A method for determining a change in concentration for each of at least two chemicals in a test sample, the method comprising:
(a) subjecting the test sample to a magnetic resonance spectroscopy sequence, wherein the at least two chemicals in the test sample have spectroscopic components at substantially the same chemical shift;
(b) acquiring free induction decay signals after an echo time, tₑ;
(c) using the free induction decay signals to determine a total intensity S(tₑ) of the spectroscopic components at the chemical shift;
(d) determining a reference intensity S_{ref}(tₑ) at the echo time tₑ;
(e) determining at least one relationship between each of the at least two chemicals;
(f) determining a relaxation time constant Tᵢ for each of the at least two chemicals; and
(g) determining the changes in the concentrations of each of the at least two chemicals relative to the reference intensity S_{ref}(tₑ) using the reference intensity S_{ref}(tₑ), the intensity S(tₑ), the at least one relationship between each of the at least two chemicals, and the relaxation time constant Ti for each of the at least two chemicals.

21. The method of claim 20 wherein the step of determining the changes in the concentrations of each of the at least two chemicals employs the relationship
$$Δ S ( {t}_{e} ) = {\sum }_{i = 1}^{n} Δ {S}_{i} {ⅇ}^{- {t}_{e} / {T}_{i}} ,$$
, wherein
ΔS(tₑ)=S(tₑ)−S_{ref}(tₑ);
n is the number of the at least two chemicals;
ΔSᵢ is a change in the spectroscopic component of each chemical at the chemical shift from a known reference value, Sᵢ^{ref} to a value, Sᵢ, such that ΔSᵢ=Sᵢ−Sᵢ^{ref};
Sᵢ₊₁ is a function of Sᵢ for i=1 to n−1, and
Sᵢ is a function f of Cᵢ, wherein Cᵢ is the concentration of each chemical, such that ΔCᵢ, the change in concentration, is given by the inverse off according to the equation:
ΔCᵢ=f⁻¹(ΔSᵢ).

22. The method of claim 20, wherein the test sample is tissue.

23. The method of claim 22, wherein the test sample is human brain tissue.

24. The method of claim 20, wherein one of the at least two chemicals is a phosphorylated form of another of the at least two chemicals.

25. The method of claim 24, wherein the two chemicals are creatine and phosphocreatine.

26. The method of claim 25, wherein the Tᵢ of phosphocreatine is between about 115 and 170 milliseconds and the Tᵢ of creatine is between about 255 and 335 milliseconds.

27. The method of claim 20, wherein the magnetic resonance spectroscopy sequence is an one-dimensional magnetic resonance spectroscopy sequence.

28. The method of claim 20, wherein the magnetic resonance spectroscopy sequence is a ¹H magnetic resonance spectroscopy sequence.

29. The method of claim 20, wherein the magnetic resonance spectroscopy sequence is a localized point-resolved spectroscopy sequence.

30. The method of claim 20, wherein the chemical shift is at about 3.08 ppm.

31. The method of claim 20, wherein the relaxation time constant, Tᵢ, is a time constant for T₂ relaxation.

32. The method of claim 20, wherein the relaxation time constant, Tᵢ, is a time constant for T₂ relaxation.

33. A method of diagnosis comprising using one or more of the chemical concentrations of claim 20 to diagnose a metabolic disorder.

34. A method of assessing response of cocaine-dependent subjects to treatment using one or more of the chemical concentrations of claim 20.

35. A method of determining changes in concentration for each of a number of chemicals in a test sample, the method comprising:
(a) subjecting the test sample to first and second magnetic resonance spectroscopy sequences at t₁ and t₂ respectively, wherein at least two of the chemicals in the test sample have spectroscopic components at substantially the same chemical shift;
(b) acquiring free induction decay signals for the first magnetic resonance spectroscopy sequence after an echo time, tₑ;
(c) using the free induction decay signals for the first magnetic resonance spectroscopy sequence to determine a total intensity S(t₁,tₑ) of the spectroscopic components at the chemical shift;
(d) acquiring free induction decay signals for the second magnetic resonance spectroscopy sequence after an echo time, tₑ;
(e) using the free induction decay signals for the second magnetic resonance spectroscopy sequence to determine the total intensity S(t₂,tₑ) of the spectroscopic components at the chemical shift;
(f) determining at least one relationship between each of the at least two chemicals;
(g) determining a relaxation time constant Tᵢ for each of the at least two chemicals; and
(h) determining the changes in the concentrations of each of the at least two chemicals between t₁ and t₂ using S(t₁,tₑ), S(t₂,tₑ), the at least one relationship between each of the at least two chemicals, and the relaxation time constant Tᵢ for each of the at least two chemicals.

36. The method of claim 35 wherein the step of determining the changes in the concentrations of each of the at least two chemicals employs the relationship
$$Δ S ( {t}_{e} ) = {\sum }_{i = 1}^{n} Δ {S}_{i} {ⅇ}^{- {t}_{e} / {T}_{i}} ,$$
, wherein
ΔS(tₑ)=S(t₂,tₑ)−S(t₁,tₑ);
n is the number of the at least two chemicals;
ΔSᵢ is a change in a contribution of each chemical to the intensity of the resonance peak,
Sᵢ(t), from a known first value at t₁ to a second value at t₂ such that ΔSᵢ=Sᵢ(t₂)−Sᵢ(t₁);
Sᵢ₊₁ is a function of Sᵢ for i=1 to n−1; and
Sᵢ is a function f of Cᵢ, wherein Cᵢ is the concentration of each chemical, such that ΔCᵢ, the change in concentration, is given by the inverse off according to the equation:
ΔCᵢ=f⁻¹(ΔSᵢ).

37. The method of claim 35, wherein the two chemicals are creatine and phosphocreatine.

38. The method of claim 37, wherein the chemical shift is at about 3.08 ppm.

39. The method of claim 35, wherein the magnetic resonance spectroscopy sequence is an 1H magnetic resonance spectroscopy sequence.

40. The method of claim 35, wherein the relaxation time constant, Tᵢ, is a time constant for T₂ decay.

41. The method of claim 40, wherein the Tᵢ of phosphocreatine is between about 115 and 170 milliseconds and the Tᵢ of creatine is between about 255 and 335 milliseconds.

42. The method of claim 35, wherein a stimulus is applied to an organism comprising the test sample.

43. The method of claim 42, wherein the stimulus is visual, auditory, or pharmaceutical.

44. A magnetic resonance spectroscopy apparatus comprising:
(a) means for subjecting a test sample comprising two different chemicals to a magnetic field;
(b) means for subjecting the test sample to an RF excitation pulse to excite nuclei in the test sample;
(c) means for acquiring data from excited nuclei in the test sample, wherein the two different chemicals in the test sample have spectroscopic components at substantially the same chemical shift; and
(d) means for processing the acquired data to obtain concentration information about each of the two chemicals, wherein the processing means employs information about a magnetic resonance relaxation property of each of the two chemicals.

45. A magnetic resonance spectroscopy data processor comprising:
(a) an input for magnetic resonance spectroscopy data; and
(b) a calculator for obtaining concentration information about each of two different chemicals that have spectroscopic components at substantially the same chemical shift in the magnetic resonance spectroscopy data, wherein the calculator employs information about a magnetic resonance relaxation property of each of the two chemicals.

46. A computer-readable storage medium comprising a program that is used by a processor to:
(a) receive magnetic resonance spectroscopy data; and
(b) obtain concentration information about each of two different chemicals that have spectroscopic components at substantially the same chemical shift in the magnetic resonance spectroscopy data by employing information about a magnetic resonance relaxation property of each of the two chemicals.
